(19) United States
(12) Patent Application Publication
(10) Pub. No.: US 2007/0072304 A1
(43) Pub. Date: Mar. 29, 2007

(54) System and method for identifying biological growth media

(75) Inventors: James R. Kraemer, Santa Fe, NM (US); David P. Vernon, Tucson, AZ (US)

Correspondence Address: HOFFMAN, WARNICK & D'ALESSANDRO LLC, 75 STATE ST, 14 FL, ALBANY, NY, 12207, US

(73) Assignee: International Business Machines Corporation, Armonk, NY (US)

(21) Appl. No.: 11/235,452

(22) Filed: Sep. 26, 2005

(51) Int. Cl. G06F 19/00; G01N 33/50

(52) U.S. Cl. 436/86; 702/19

(57) ABSTRACT

A system and method for encoding and then identifying a growth media from among a plurality of different growth media. A method is disclosed that includes: providing a set of tagging agents that are distinct from each of the different growth media; adding to each different growth media a unique subset of the tagging agents selected from the set of tagging agents in order to encode each different growth media; and determining an identity of one of the different growth media by detecting an existence of each tagging agent in the set of tagging agents.

### BACKGROUND OF THE INVENTION

1. Technical Field

The present invention relates generally to identifying biological substances, and more specifically relates to a system and method for identifying biological growth media through the use of inorganic tagging agents readable by an automated processing device.

2. Related Art

Biological growth media “growth media” is manufactured with a series of standard inputs. There are tens of thousands of different types of growth media with slightly varying biological inputs. Each firm in the industry provides a large number of growth media to support the growth of different cells and bacteria. For large scale users such as government, research universities, and large hospitals, growth media is sold in large bulk containers to scientific labs, and each lab prepares the growth media and places the growth media into growth plates for use in experiments. Depending on the specific growth media selected for use in the experiment, significantly different results can occur. Accordingly, the lab must be extremely careful to accurately mark each plate with the specific type of growth media in the plate. Unfortunately, when handling large numbers of growth plates with different types of growth media, mistakes can easily be made. Such mistakes can have significant consequences and, e.g., lead to the incorrect interpretation of results with very negative outcomes for patients, or lead to a national disaster in the area of homeland security.

One approach to eliminating such mistakes is to provide a method for directly detecting the exact type of growth media being used in an experiment. By providing such a method, a lab could make sure that they used the correct type of growth media in an experiment. Unfortunately, given the large number of different types of growth media, no such methodology exists that can ensure complete accuracy. Accordingly, a need exists for a methodology for identifying different biological growth media.

### SUMMARY OF THE INVENTION

The present invention addresses the above-mentioned problems, as well as others, by using multiple inorganic agents (i.e., tagging agents) to encode biological growth media and then provides an automated device that can positively identify the growth media by examining the inorganic agents. The use of multiple inorganic agents allows for the encoding of a large number of growth media with a limited number of tagging agents. For example, eight tagging agents allows for the completely accurate identification of 255 growth media. Twenty tagging agents allows for the identification of over one million distinct growth media. Simply stated, using this methodology, n tagging agents allows for encoding and identification of 2ⁿ−1 different types of growth media.

In a first aspect, the invention provides a method for identifying a growth media from among a plurality of different growth media, comprising: providing a set of tagging agents that are distinct from each of the different growth media; adding to each different growth media a unique subset of the tagging agents selected from the set of tagging agents in order to encode each different growth media; and determining an identity of one of the different growth media by determining which tagging agents in the set of tagging agents exists in the growth media.

In a second aspect, the invention provides a system for encoding a plurality of different growth media, comprising: a database that includes each of the plurality of different growth media and an associated tagging code for each different growth media; and a selection system for selecting a unique subset of tagging agents from a fixed set of tagging agents for each of the plurality of different growth media, wherein the selection system determines the unique subset of tagging agents for a selected growth media by examining the database to determine the associated tagging code for the selected growth media.

In a third aspect, the invention provides a system for identifying a growth media from among a plurality of different growth media, wherein each of the different growth media are encoded with a subset of tagging agents selected from a fixed set of tagging agents, the system comprising: a detection system for detecting a subset of tagging agents present in a growth media; a database that includes each of the plurality of different growth media and an associated tagging code for each different growth media; and an identification system that determines an identity of a growth media by comparing the subset of tagging agents with the associated tagging codes stored in the database.

In a fourth aspect, the invention provides a method for deploying an application for identifying a growth media from among a plurality of different growth media, wherein each of the different growth media are encoded with a subset of tagging agents selected from a fixed set of tagging agents, comprising: providing a computer infrastructure being operable to: input a subset of tagging agents present in a growth media; and determine an identity of a growth media by comparing the subset of tagging agents with associated tagging codes stored in a database that includes each of the plurality of different growth media and an associated tagging code for each different growth media

By eliminating human input from the identification of growth media, this process allows labs to: (1) increase accuracy of results, (2) cut the staff skill level required, and (3) speed up the analysis. In a time critical medical or public health situation, speed and accuracy could provide life saving value. In day to day medical operations, this invention could cut medical errors and increase productivity.

### BRIEF DESCRIPTION OF THE DRAWINGS

These and other features of this invention will be more readily understood from the following detailed description of the various aspects of the invention taken in conjunction with the accompanying drawings in which:

FIG. 1 depicts a system for tagging biological growth media in accordance with the present invention.

FIG. 2 depicts a system for identifying a biological growth media in accordance with the present invention.

### DETAILED DESCRIPTION OF THE INVENTION

As noted above, the present invention utilizes tagging agents for identifying and distinguishing a biological growth media from among a plurality of possible biological growth media. The identity of the biological growth media can be determined by looking for the existence/non-existence of various tagging agents, which were added to the biological growth media, e.g., during a manufacturing process. While the embodiments described herein relate to a process for identifying biological growth media, it should be understood that the invention could be applied to tagging/detecting any type of biological media.

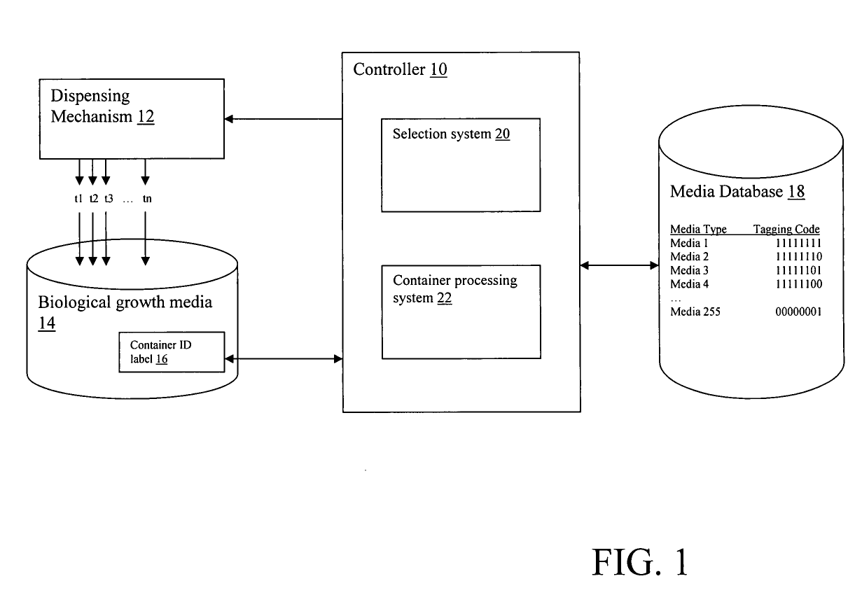

FIG. 1 depicts an illustrative system for tagging a biological growth media 14. The system operates by first providing a fixed set of n tagging agents, tl, t2, t3 . . . tn. Each of the n tagging agents comprises a unique inorganic compound that is non-deleterious, inert, and not inherently part of any of the plurality of biological growth media 14 being tagged. A dispensing mechanism 12 may be utilized to automatically add tagging agents into the biological growth media 14. Depending on the specific type of biological growth media 14 being tagged (e.g., Media 1, Media 2, etc.), a different subset (i.e., combination) of the n tagging agents are added.

In this example, a controller 10 is utilized to control the dispensing of tagging agents from the dispensing mechanism 12. In this illustrative embodiment, controller 10 includes: (1) a selection system 20 for selecting the subset of tagging agents to be added for a particular biological growth media 14, and (2) a container coding system 22.

Selection system 20 determines the subset of tagging agents for a given biological growth media 14 (i.e., “media type”) based on an n digit binary code (“tagging code”) associated with the media type. In this example, tagging codes for each media type are stored in a media database 18, and each unique media type is given a unique code. In this embodiment, tagging codes are implemented as an n digit binary number; however it should be understood that other equivalent representations may be utilized as well. In this case, each tagging code comprises an 8 digit binary number 00000000-11111111, which allows for the encoding of 255 different media types. (Note that while there are technically 2⁸ or 256 possible codes for this example, the use of 00000000, meaning no tagging agents are used, would not be recommended.) Each binary digit position “dl d2 d3 d4 d5 d6 d7 d8” in the tagging code corresponds to a different tagging agent “tl t2 t3 t4 t5 t6 t7 t8.” Thus, for a process that utilizes eight tagging agents, each tagging code comprises eight binary digits, and each digit may comprise a “1” or a “0.” A value of “1” in a given digit position indicates that the corresponding tagging agent should be added, and a value of “0” indicates that the corresponding tagging agent should not be added. Accordingly, n tagging agents allows for 2ⁿ−1 unique tagging codes.

For example, a tagging code of 10101010 would indicate that tagging agents, tl, t3, t5 and t7 should be added, and t2, t4, t6 and t8 should not be added. A tagging code of 00000001 indicates that only tagging agent t8 should be added. Accordingly, selection system 20 can readily translate each unique tagging code into a unique subset of tagging agents. Obviously, the number of tagging agents used (i.e., size of the code) can be adjusted to meet the needs of a particular scheme. For instance, while eight tagging agents allows for encoding 255 media types, 20 tagging agents would allow for encoding over a million media types.

A container coding system 22 maybe utilized to read identification information from a container ID label 16 in order to facilitate the tagging agent selection process. For instance, container coding system 22 could read/process the name of the biological growth media, a serial number or bar code, or an actual tagging code from the label 16. Based on that information, selection system 20 could determine the subset of tagging agents to use for the media contained therein. Alternatively, container coding system 22 could instead generate a container ID label 16 with the tagging code used for the biological growth media 14 for future reference.

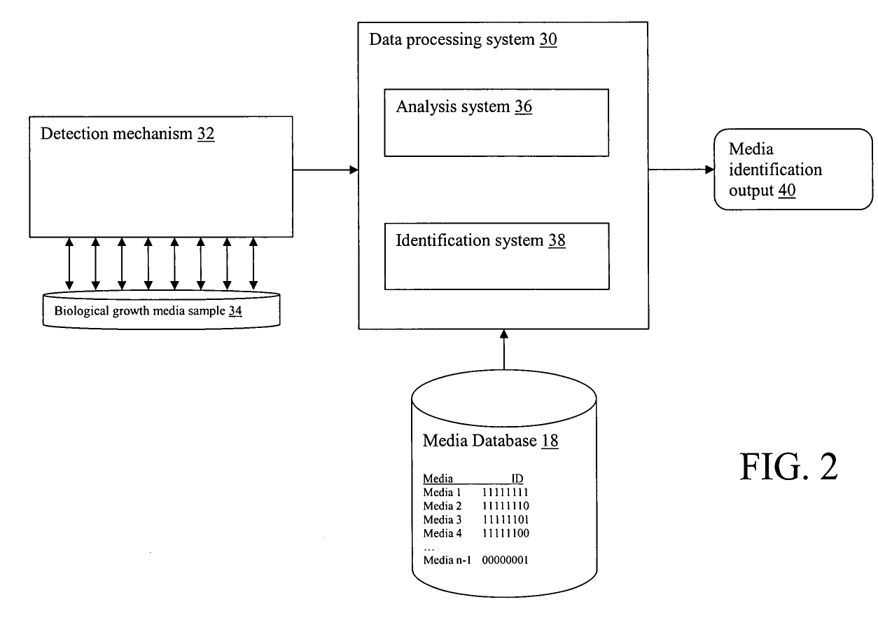

Referring now to FIG. 2, an illustrative system for identifying a biological growth media sample 34 (e.g., which resides on a growth plate) that was tagged in a manner described above is shown. The system of FIG. 2 includes a detection mechanism 32, a data processing system 30, and a media database 18. In this example, detection mechanism 32 and an analysis system 36 are provided to detect the existence of any of the fixed set of n tagging agents which may have been used to encode the sample 34. Any number of different methods can be used to detect the tagging agents. One method would be to expose the sample 34 to specific light frequencies to detect any of the n tagging agents (using e.g., a frequency analysis system, an infrared system, a visible light detection system, an ultraviolet light detection system, etc.). Alternately, the sample 34 could be processed by a mass spectrometer to identify the existence of any of the n tagging agents, which would appear as unique signatures (e.g., spikes) in a graphical set of output data. In such a case, analysis system 36 would analyze the output data and determine which of the n tagging agent's signatures appear in the output data. Identification system 38 can then compare the subset of tagging agents that are present in the media sample 34 with the tagging codes in the media database 18 to determine the identity of the biological growth media sample 34, and then provide a media identification output 40. Note that the compare step may require that subset of tagging agents that are identified as present be converted to a tagging code.

Thus, in a system that used eight tagging agents, if detection mechanism 32 and analysis system 36 determined that the sample 34 included tagging agents t1-t6 (and did not include tagging agents t7 and t8), then by examining media database 18, identification system 38 could readily ascertain that the sample 34 consisted of “Media 4,” which has an associated tagging code of 11111100. Accordingly, as long as the biological growth media sample 34 was tagged using a scheme described above, and access was provided to a media database 18 that associated media types with a tagging code, the sample's identity can be ready determined.

In general, controller 10 and data processing system 30 could be implemented using any type of computer system, and could be implemented as part of a client and/or a server. Such a computer system generally includes a processor, input/output (I/O), memory, and a bus. The processor may comprise a single processing unit, or be distributed across one or more processing units in one or more locations, e.g., on a client and server. Memory may comprise any known type of data storage and/or transmission media, including magnetic media, optical media, random access memory (RAM), read-only memory (ROM), a data cache, a data object, etc. Moreover, memory may reside at a single physical location, comprising one or more types of data storage, or be distributed across a plurality of physical systems in various forms.

I/O may comprise any system for exchanging information to/from an external resource. External devices/resources may comprise any known type of external device, including a monitor/display, speakers, storage, another computer system, a hand-held device, keyboard, mouse, voice recognition system, speech output system, printer, facsimile, pager, etc. Bus provides a communication link between each of the components in the computer system and likewise may comprise any known type of transmission link, including electrical, optical, wireless, etc. Although not shown, additional components, such as cache memory, communication systems, system software, etc., may be incorporated into computer system.

Access to such a computer system may be provided over a network such as the Internet, a local area network (LAN), a wide area network (WAN), a virtual private network (VPN), etc. Communication could occur via a direct hardwired connection (e.g., serial port), or via an addressable connection that may utilize any combination of wireline and/or wireless transmission methods. Moreover, conventional network connectivity, such as Token Ring, Ethernet, WiFi or other conventional communications standards could be used. Still yet, connectivity could be provided by conventional TCP/IP sockets-based protocol. In this instance, an Internet service provider could be used to establish interconnectivity. Further, as indicated above, communication could occur in a client-server or server-server environment.

Media database 18 could likewise be implemented in any fashion, e.g., as a relational database, a table, a data object, a flat file, etc. Media database 18 could also reside in a single location or be distributed and/or accessed over a network. Obviously, the specific format and arrangement of the information in media database 18 could vary depending on the particular implementation.

It should be appreciated that the teachings of the present invention could be offered as a business method on a subscription or fee basis. For example, a computer system comprising a data processing system 30 could be created, maintained and/or deployed by a service provider that offers the functions described herein for customers. That is, a service provider could offer to provide media type identification based on inputs from a detection mechanism 32 as described above.

It is understood that the systems, functions, mechanisms, methods, engines and modules described herein can be implemented in hardware, software, or a combination of hardware and software. They may be implemented by any type of computer system or other apparatus adapted for carrying out the methods described herein. A typical combination of hardware and software could be a general-purpose computer system with a computer program that, when loaded and executed, controls the computer system such that it carries out the methods described herein. Alternatively, a specific use computer, containing specialized hardware for carrying out one or more of the functional tasks of the invention could be utilized. In a further embodiment, part of all of the invention could be implemented in a distributed manner, e.g., over a network such as the Internet.

The present invention can also be embedded in a computer program product, which comprises all the features enabling the implementation of the methods and functions described herein, and which—when loaded in a computer system—is able to carry out these methods and functions. Terms such as computer program, software program, program, program product, software, etc., in the present context mean any expression, in any language, code or notation, of a set of instructions intended to cause a system having an information processing capability to perform a particular function either directly or after either or both of the following: (a) conversion to another language, code or notation; and/or (b) reproduction in a different material form.

The foregoing description of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of this invention as defined by the accompanying claims.

1. A method for identifying a growth media from among a plurality of different growth media, comprising:
providing a set of tagging agents that are distinct from each of the different growth media;
adding to each different growth media a unique subset of the tagging agents selected from the set of tagging agents in order to encode each different growth media; and
determining an identity of one of the different growth media by determining which tagging agents in the set of tagging agents exists in the growth media.

2. The method of claim 1, wherein the tagging agents are inorganic agents.

3. The method of claim 1, wherein each unique subset of tagging agents is selected according to a predetermined tagging code established for an associated growth media.

4. The method of claim 3, wherein the predetermined tagging code for each different growth media is stored in a database.

5. The method of claim 1, wherein up to 2ⁿ−1 different growth media can be encoded with n tagging agents.

6. The method of claim 1, wherein the step of determining which tagging agents in the set of tagging agents exist in the growth media includes using a mass spectrometer to analyze one of the different growth media.

7. The method of claim 1, wherein the step of determining an identity of one of the different growth media includes referencing a database that includes each different growth media and an associated tagging code.

8. A system for encoding a plurality of different growth media, comprising:
a database that includes each of the plurality of different growth media and an associated tagging code for each different growth media; and
a selection system for selecting a unique subset of tagging agents from a fixed set of tagging agents for each of the plurality of different growth media, wherein the selection system determines the unique subset of tagging agents for a selected growth media by examining the database to determine the associated tagging code for the selected growth media.

9. The system of claim 8, further comprising a dispensing mechanism for adding the unique subset of tagging agents associated with the selected growth media into the selected growth media.

10. The system of claim 8:
wherein the tagging code comprises an n digit binary number; and
wherein each binary digit is associated with a different tagging agent; and
wherein a value assigned to each binary digit determines whether an associated tagging agent is to be added.

11. The system of claim 8, wherein the tagging agents are inorganic agents.

12. The system of claim 8, wherein up to 2ⁿ−1 different growth media can be encoded with n tagging agents.

13. The system of claim 8, further comprising a container coding system that can process information obtained from a label on the growth media for use in selecting the unique tagging agent.

14. The system of claim 8, further comprising a container coding system that can generate a label for the growth media that includes the tagging code used for the growth media.

15. A system for identifying a growth media from among a plurality of different growth media, wherein each of the different growth media are encoded with a subset of tagging agents selected from a fixed set of tagging agents, the system comprising:
a detection system for detecting a present subset of tagging agents in a growth media;
a database that includes each of the plurality of different growth media and an associated tagging code for each different growth media; and
an identification system that determines an identity of a growth media by comparing the present subset of tagging agents with the associated tagging codes stored in the database.

16. The system of claim 15, wherein the detection system comprises a mass spectrometer.

17. The system of claim 15, wherein the detection system is selected from the group consisting of a frequency analysis system, an infrared system, a visible light detection system, and an ultraviolet light detection system.

18. The system of claim 15, wherein the identification system translated the present subset of tagging agents into a tagging code.
